(19) United States
(12) Patent Application Publication
(10) Pub. No.: US 2026/0265785 A1
(43) Pub. Date: Sep. 10, 2026

(54) DEVICE AND METHOD FOR CAPTURING ATMOSPHERIC POLLUTANTS

(71) Applicant: LIFE WORK SERVICOS ESPECIALIZADOS LTDA, SAO PAULO (BR)

(72) Inventor: CICERO FARIAS SILVA, Sao Paulo (BR)

(21) Appl. No.: 19/670,504

(22) Filed: May 7, 2026

Related U.S. Application Data
(63) Continuation-in-part of application No. PCT/BR2024/050206, filed on May 21, 2024.

(30) Foreign Application Priority Data
Apr. 29, 2024 (BR) 1020240084543

(51) Int. Cl. C12P 3/00; C12M 1/00; C12R 1/89

(52) U.S. Cl. CPC C12P 3/00; C12M 21/02; C12R 2001/89

(57) ABSTRACT

The present invention deals with a need in the field of sustainability and involves a sustainable alternative for the improvement of air quality, and with an emphasis on the capture of carbon dioxide and carbon monoxide using aquatic organisms, microalgae and cyan bacteria.

### CROSS-REFERENCE TO RELATED APPLICATION

This application claims benefit from and is a bypass continuation-in-part of International Application No. PCT/BR2024/050206, filed May 21, 2024, which claims the benefit of Brazilian Patent Application No.: BR 1020240084543, filed Apr. 29, 2024, the contents of which are hereby incorporated by reference.

### TECHNICAL FIELD

The present invention and process was created from the need for a sustainable alternative for the improvement of air quality and has an emphasis on the capture of carbon dioxide and carbon monoxide using aquatic organisms, microalgae and cyan bacteria.

### BACKGROUND

Around 54% of the oxygen generated in the atmosphere happens through the process of photosynthesis in an aqueous medium. Our atmosphere was built over time, in a combination of elements and physicochemical circumstances that led to the appearance of life as it is known. Carbon Dioxide (CO₂) and Carbon Monoxide (CO) are gases resulting from these combinations, either naturally or by anthropic action, forming our atmospheric layer with other gases that are important to maintain temperature and filter unwanted solar radiation, characterized by the greenhouse effect, however economic development and the need for industrial support have caused the balance of these gases to be affected, promoting major climate change.

All living beings on the planet need oxygen for subsistence, the importance of oxygen generators is paramount for the support of life, placing photosynthetic organisms as a priority for the sustainability of the planet.

### SUMMARY

For the above-referenced reasons, the system chosen to develop the process of oxygen generation and carbon retention was aquatic and, thus, studies of photosynthesis in an aqueous medium were initiated.

Based on these studies, it was verified that photosynthetic organisms, algae, microalgae and cyan bacteria, present in fresh and salt water, could promote an efficient process with the possibility of being reproduced in closed systems.

The beginning of the work aimed to collect microalgae, in their natural habitat, for the assembly of the system and due developments, the most common and resistant species was the species Scenedesmus.

Later, pure strains were removed from the USP-SP algae bank, which provided us with two types that proved to be efficient, microalgae Chlorella and cyan bacterium Spirulina.

Within the tests and sampling of the results, the blend consisting of the micro algae Chlorella and Scenedesmus and the cyan Bacterium Spirulina, were considered the most efficient, being chosen as the ideal for the process of air purification, carbon retention and oxygen generation.

As a container to receive the process, photobioreactors were chosen, which can be built in different materials, shapes and sizes. Several forms were analyzed, always evidencing the shape, bubbling, flow and movement, gas transfer, and exposure to light energy.

The photobioreactors used throughout the experimental phases are hereinafter designated as P1, P2, and P3, each corresponding to a different scale and function within the development of the invention.

P1 refers to a first photobioreactor having an approximate working volume of 80 liters, configured for initial validation of organism performance, including efficiency of carbon retention, resistance to environmental conditions, and behavior of individual and blended microorganisms under controlled conditions.

P2 refers to a second photobioreactor having an approximate working volume of 8 liters, configured as a bench-scale system for controlled and repeatable testing of process variables, including pH, temperature, gas exchange rate, and blend stability over time.

P3 refers to a third photobioreactor having an approximate working volume of 800 liters, configured as a large-scale system for evaluating scalability, integrated system performance, and real-world operational efficiency, including continuous monitoring through sensors and full system integration.

In aquatic environments, seas and rivers, the exchanges between atmospheric gases and the system are through surface contact, water flower and the atmosphere, waves or whirlwind promoted by the movement of the waters and promote the saturation of the gases to the medium, which have been studied to develop the saturation of the process through bubbles for saturation of the photobioreactor.

The volume of gases inserted is relative to the volume and photosynthesis capacity of the organisms present. The bubbling system consists of a micro compressor, and a bubbling element, created to homogenize the micro bubbles constantly and efficiently.

The variables chosen to control the process were, gas exchange through bubbling, volume of gases inserted into the system, temperature, pH, population density of organisms, food and light energies for photosynthesis.

In all photobioreactors (P1, P2, and P3), the air purification process comprises: (i) introduction of atmospheric air into an aqueous medium containing a blend of microalgae and/or cyanobacteria; (ii) dispersion of said air in the form of microbubbles generated by a microcompressor; (iii) dissolution and availability of carbon dioxide (CO₂) and carbon monoxide (CO) in the aqueous medium; (iv) photosynthetic assimilation of said gases by the microorganisms under illumination within the wavelength range of 400 nm to 700 nm; and (v) release of oxygen (O₂) and discharge of treated air with reduced carbon content.

The process further includes continuous or periodic monitoring of gas concentration, pH, temperature, and biomass density, allowing adaptive control of the system to maintain optimal carbon retention efficiency.

### BRIEF DESCRIPTION OF THE DRAWINGS

Other objects and features of the present invention will become apparent from the detailed description considered in connection with the accompanying drawings, which disclose several embodiments of the invention. It is to be understood that the drawings are to be used for the purpose of illustration only and not as a definition of the limits of the invention.

FIG. 1 illustrates the location of the lake where the first microalgae sample was collected (image courtesy: Google Earth).

FIG. 2 shows the withdrawal of water, to verify the aqueous medium and its properties.

FIG. 3 illustrates Scenedesmus, the first organism studied.

FIG. 4 shows the bubbler.

FIG. 5 reveals the sensors for monitoring, and evidence of the efficiency of CO2 retention, where (g)—monitoring of the output gases and (h)—monitoring of the input gases.

FIG. 6 illustrates control images of the microalgae, where (a) Chlorella; and Cyan bacterium and (b) Spirulina.

FIG. 7 illustrates the pots for blending development and testing with and without bubbling.

FIG. 8 test containers from the research carried out.

FIGS. 9, 10, 11 illustrate variations of the photobioreactors studied by the structure created from the research.

FIG. 12 shows a variation of one of the structural versions with a human model next to it to scale height.

FIG. 13 illustrates a perspective of the variation of one of the structural versions, where (f) is the reservoir, (d) is the seat for accommodating people and (e) there are 5V USB outputs for charging cell phones or electronic devices.

FIGS. 14 to 27 are graphs of saturation and temperature measurements with microalgae on different dates.

### DETAILED DESCRIPTION

Materials and equipment used for research-PH meter, Magnifying glass 500×, Microscope 1500×, Camera for microscope, special oil lenses, analytical balance, micro compressor, beaker, Imhoff cone, Glass jars 4 Lts, conductivity meter, tweezers, pipette, glassware, computer, manual magnifying glass, buffer solution, vitamin compound for aquatic organisms, freezer, heater, oven, bubblers, general glassware, hoses and hydraulic material, lamps, LED lamps, 8 lts, 80 Lts, 800 lts photobioreactor, camera, printer, 20 lts gallons, 50 lts gallons, Greenhouse, tweezers, IT equipment, flame spectrophotometer, CO₂ sensors, photometers, 3 computers, slides for microscopic evaluation, pumps for collection, air quality samplers.

All carried out from the following steps to study and select microalgae efficient in carbon retention through photosynthesis in an aquatic environment:
i. Studies on microalgae aiming at the selection of organisms efficient in carbon retention by photosynthesis in an aquatic environment.
ii. Investigation of forms of photobioreactors for the development of efficient carbon retention processes.
iii. Analysis of the best ways to provide radiant energy for the photosynthesis process in photobioreactors.
iv. Evaluation of the best approach to gas exchange of carbon with the environment and exposure to organisms.
v. Verification of the best practices for the circulation of the aqueous medium by means of bubbling, aiming at the homogenization of the environment.
vi. Study of the relationship between terrestrial and aquatic organisms to develop comparisons on the effectiveness in carbon retention between these groups.
vii. Development of more suitable forms of photobioreactors for process optimization.
viii. Data collection to demonstrate the flows and effectiveness of the carbon retention process.
ix. Conducting efficacy tests to validate the approaches adopted.
X. Development of process monitoring equipment for continuous monitoring.
xi. Determination of the variables that interfere in the carbon retention process.
xii. Elaboration of procedures and materials for the systematic monitoring of studies over time.

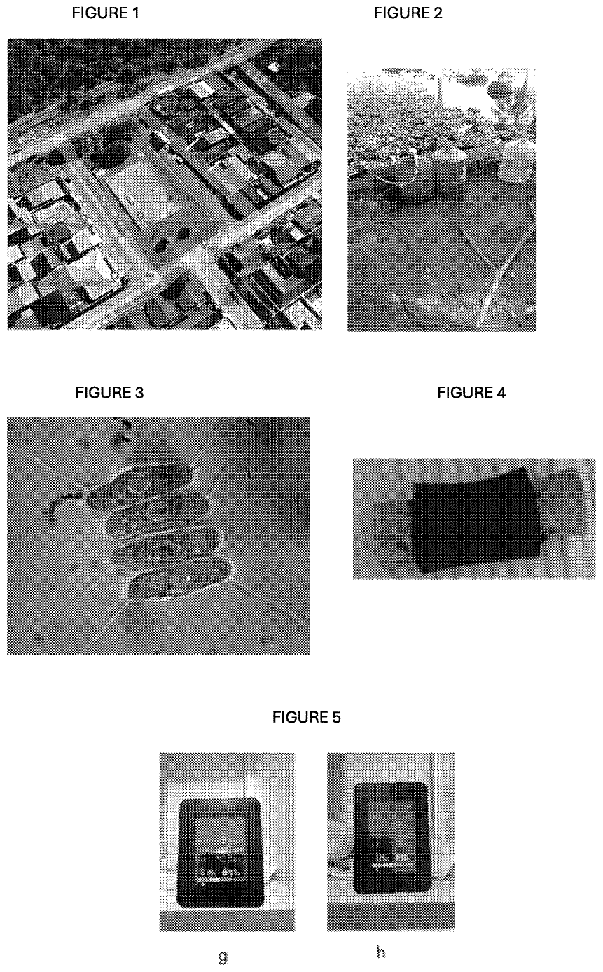

Search for locations for possible collection of materials for possible evaluations and tests. (FIGS. 1, 2). Verification of strains present in the first collections (FIG. 3).

From phase I, daily from Monday to Friday, pH, temperature, humidity, VOC were measured and an evaluation by microscopy with a camera for external imaging was carried out, on the organisms present in the aqueous medium of the experiment, for recognition and evaluation of the efficiency, amount of biomass and progress of the process.

In Phase II, the collected material was separated by removing small fish and apparent dirt, and separated by pots for verification; pH determination and assembly of controls for verification and identification of organisms were carried out. The process of evaluation of organisms and training for multiplication and viability of aqueous medium (use of common drinking water) has begun. In addition, the assembly of saturation of the medium was carried out through bubbling with different bubblers.

In phase III, 4-liter volume reservoirs were used. With different pH and constant temperature, six units were used to verify the preservation capacity and verification of the life cycle and possible means of feeding and carbon retention. Sensors were also installed to monitor effective carbon retention and freeze and thaw samples for endurance tests (FIG. 5).

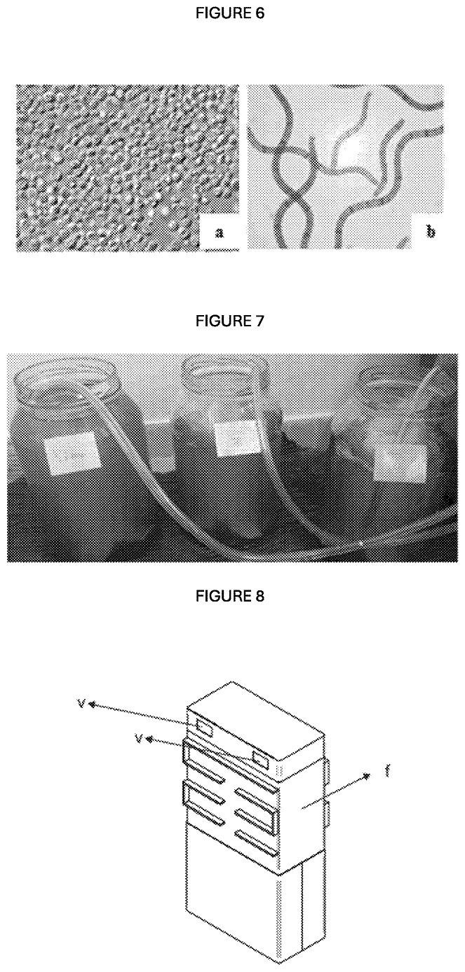

In phase IV, a microalgae sample was taken from the USP SP Chlorella and Spirulina Algae Bank, 10 ml of solution of each (FIG. 6). In this phase, the multiplication of the microalgae Chlorella and cyan bacterium Spirulina were initiated. To this end, a preparation of the 80 Lts “P1” Photobioreactor was carried out, to receive the first multiplied material at the Research Center, from the micro algae Scenedesmus for efficiency and resistance tests to the common water medium.

From phase IV onwards, only common water was used for the experiments to adapt to the proposed process (FIG. 7).

Phase V had its beginning of pH control with buffer solution, beginning of control on P1 efficiency, Scenedesmus, control of the appearance of protozoa, monitoring of the multiplication of Spirulina and Chlorella, verification of the pH variable in the multiplication of all organisms.

The VI phase initiates the composition of the blend with Scenedesmus, Spirulina and Chlorella, control of the variables that determine the best development of each organism intercropped in the blend, controls of the efficiency of P1 with blend, pH control and other variables of mutltypation and efficiency.

In phase VII the P2 project with 8 lts, as a model for daily tests of efficiency and time for maintenance of the blend, assembly of isotherms of variables, pH, temperature, humidity and volatiles (air quality controls), verification of the blend.

The Vill phase begins the tests with feeding with mineral supplement, verification of parameters, first test with the use of liquid soda to raise pH, change in the form of bubbling, multiplication of Spirulina using low pH, multiplication of Chlorella and Scenedesmus at low pH.

Phase IX determines the P3 800 lts and the blend to be used, testing multiplication system with 50 lts drums, testing individual lighting and feeding for fast multiplication, goal 500 lts in 2 weeks.

The transition from P1 to P2 and subsequently to P3 represents a progressive scaling of the same underlying process, wherein operational parameters identified in P2 are applied and validated in P1 and subsequently implemented at scale in P3.

Phase X brings the assembly of the P3 Central Life, placement of the sensors for measuring P3 efficiency, checking problems with hydraulic installation, multiplication and blending solution.

Phase XI hits the noise above the forecast, verification of the blend and efficiency, development of support blend, tests with resistant blend to pH variability.

In phase XII there are tests with alternative blend, tests to verify the efficiency of resistant blend, temperature change to verify blend, cleaning of blend, and objective monitoring of % of each algae present.

Phase XIII shows Blende for F1 presentation, follow-up of the development of 500 lts blende for P3 F1, installation, assembly and follow-up F1.

Phase XIV there is the determination of new sensors and logic for monitoring, verification and testing of the new sensors, blending monitoring and variations of Chlorella and Scenedesmus in the blend.

Phase XV shows the installation of the new monitoring and preparation of P2 for 73-hour tests, verification and measurement of the new monitoring and consolidation of 72-hour tests of blende efficiency using P2 8 lts for graphs and data.

As the blend adapted to the common aqueous medium, composed of potable, chlorinated and fluoridated water, distributed to most localities through the public network, the microalgae Scenedesmus emerged as the most resilient. Modifying its morphology, it remained active at temperatures ranging from 18 to 36° C. Although it rarely exceeded 30% of the blend's composition, the cyanobacterium Spirulina showed greater activity at higher constant temperatures, above 25° C. Due to its spiral structure, Spirulina has led to the formation of large clumps in conjunction with Scenedesmus and Chlorella, leading us to select an intermediate bubble size in order to optimize system saturation for greater efficiency.

In turn, the microalgae Chlorella started to reproduce in all temperature ranges, being able to compose up to 40% of the blend, resulting in an adaptable and balanced mixture, maintaining a constant efficiency.

The graphs showed occurrences designated as anomalies. At certain times, variations in atmospheric pressure, air movements, or a “gust” of air precursor to rain, can temporarily decrease the concentration of CO₂ at the entrance to the system. This can cause, until the “new” air is saturated, results detected by the sensor as divergent, where the input concentration is lower than the output. This phenomenon is explained by the average saturation of the incoming gas, while the process of retention or photosynthesis occurs in the already saturated medium, which rebalances itself as the outside air maintains a constancy.

The tests carried out over 72 hours corroborated this finding. The blend adjusts automatically, increasing the biomass, which, at regular intervals, requires purging by draining the liquid medium with the blend, followed by replenishment with water. The drained material can be used as biofertilizer or, through technical evaluation, for other sustainable purposes.

In addition, the new monitoring systems, in addition to being more adequate, can, through digital alerts, inform the right time for drainage, thus facilitating intervention in this process.

In the assembly process, it can be evidenced that 54% of the oxygen generated in the process happened in an aquatic environment, showing that the way to efficiency of the study would be in aquatic organisms. The organisms and all the available aquatic processes and media were studied to indicate the most effective way for air purification, with oxygen generation and carbon retention.

Thus, the present study proved that the creation of the air purifier with emphasis on the removal of atmospheric CO₂ and CO, with generation of Oxygen (O₂). And as specific objectives to evaluate the retention of atmospheric CO₂ and CO, using photosynthesizing photobioreactor using microalgae and cyan bacteria and or aquatic organisms.

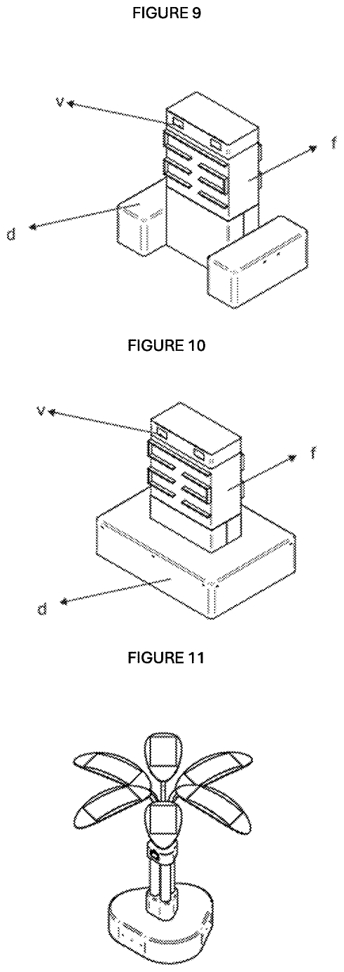

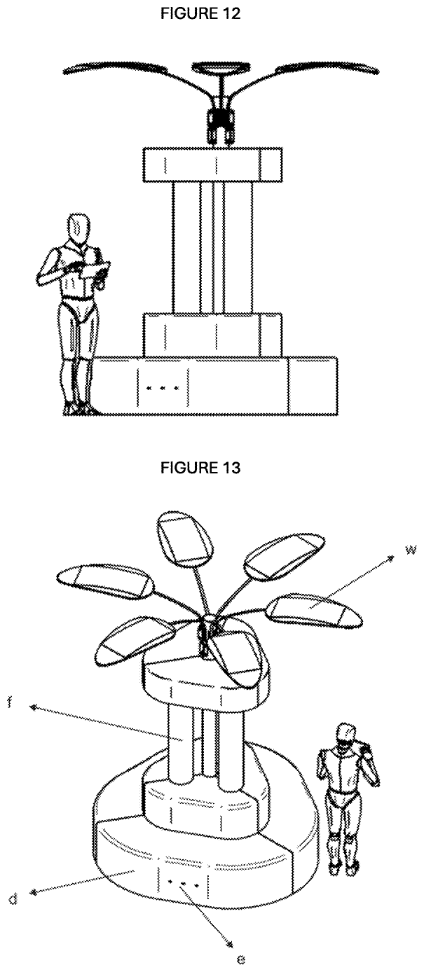

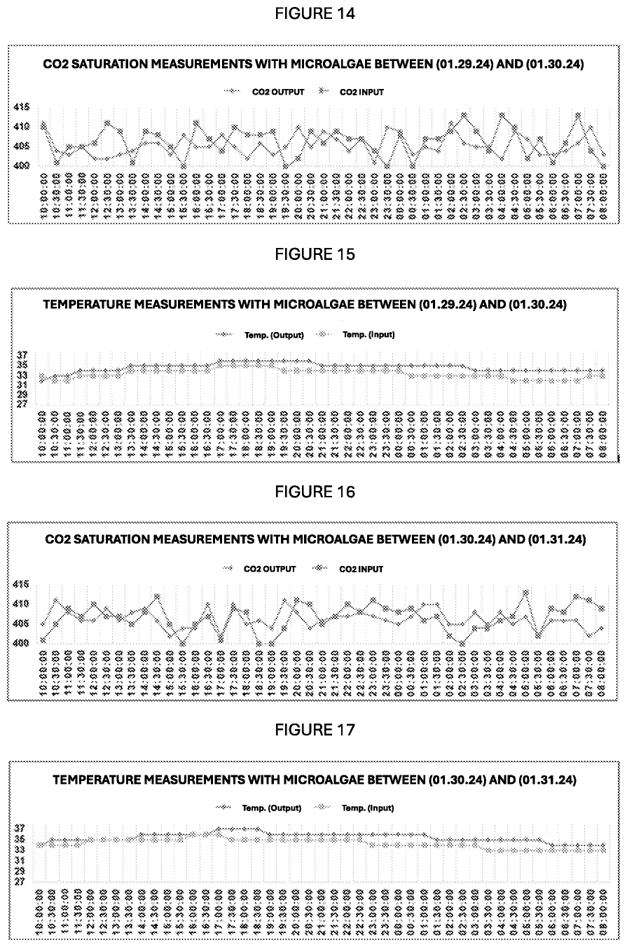

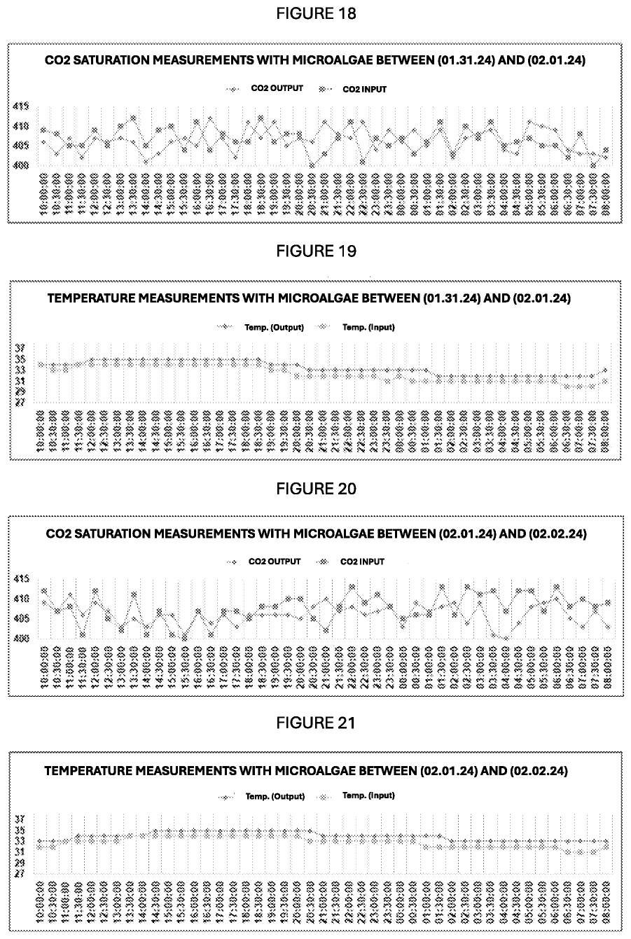

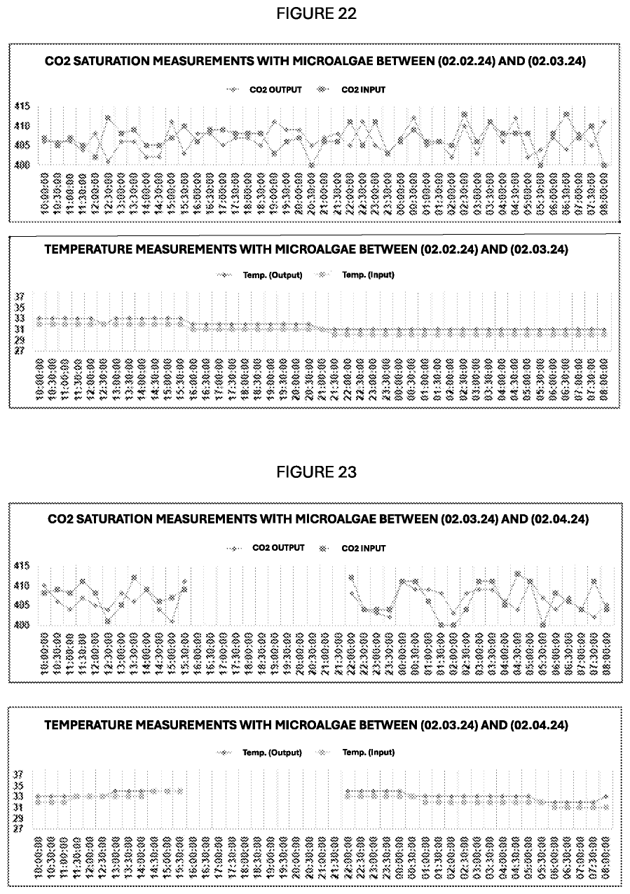

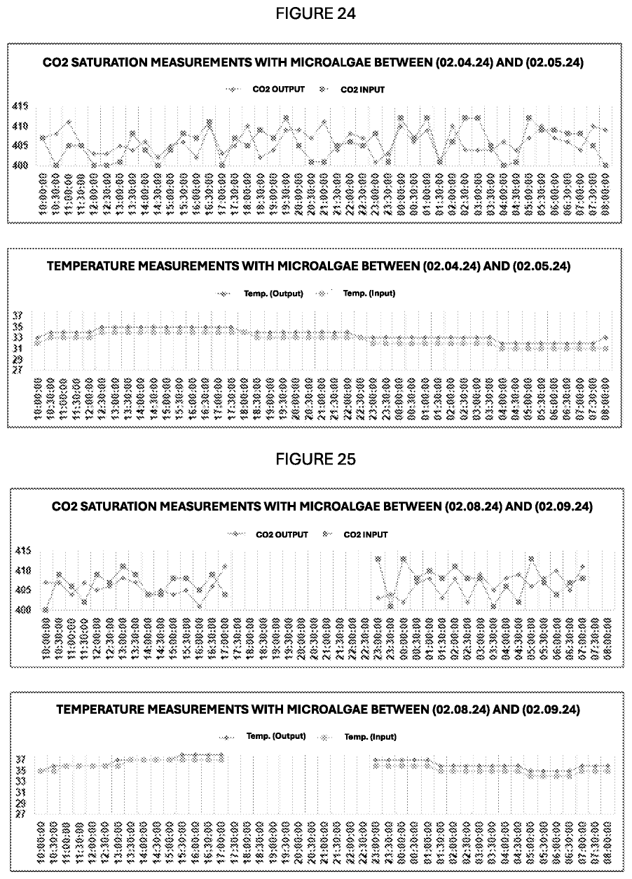

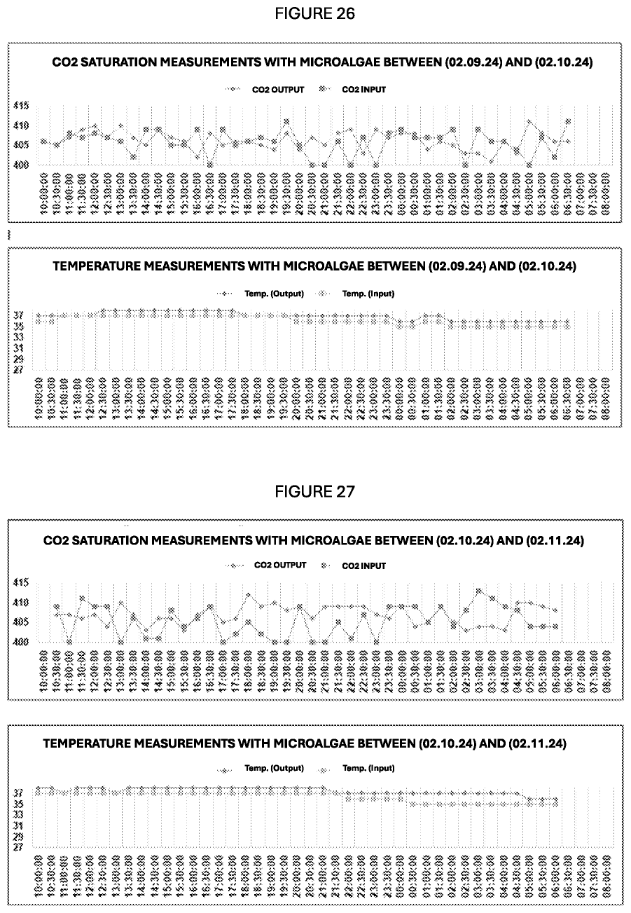

Based on the principle of this study, the structures (FIGS. 8 to 13) were created for the purification of the air, in which a purifying equipment (FIGS. 8 to 13) is described, which uses in its reservoir (f) a compound based on water and algae, which perform the process of photosynthesis, absorbing carbon dioxide and releasing oxygen, returning the air to the environment significantly with better quality. This equipment has seats (d), USB connection (e) 5V output (cellular charge), tubular structure in galvanized steel or similar material, finish in enveloped acrylic, wood or similar material. They may have displays (v) that display the amount in “parts per million (ppm)” of carbon dioxide particles in the air captured from the environment; and “parts per million (ppm)” amount of carbon dioxide particles after processing the captured air where the processed air will be returned to the environment with a smaller amount of carbon dioxide particles and also display the “parts per million (ppm)” amount of particles removed from the air captured from the environment, the ambient temperature and the relative humidity of the air. In the tree version structure (FIGS. 11 to 13) it also has blades (w) that simulate sheets in steel or similar material containing photocell plates, crystal acrylic reservoir or similar material with a capacity of approximately 400 liters.

There is a lot of efficiency in the product achieved: Each 8 liters is equivalent to a 1-ton tree (a tree to reach 1 ton takes an average of 10 years accumulates 170 kg of carbon).

The type of bubbling: Per microcompressor, capacity depends on the water column (reactor height) and amount of aqueous solution.

The type of lamp: Spectrum that corresponds to the wavelengths suitable for photosynthesis, is part of the visible light spectrum and is between 400 nm and 700 nm. Rectangular models can have from 8 liters to 800 liters.

The tree type model can have a structure for up to 400 lts tubular and containers with operation methodology: Aqueous medium with a blend of 3 organisms with saturation by micro bubbles using micro compressor, 24-hour lighting LED lamps 10 w visible light spectrum between 400 nm and 700 nm.

Photobioreactor: Container that contains a certain volume built according to the need and technical determination, with lighting according to predetermined light frequency, maintains inside an aqueous medium with a solution of microalgae and cyan photosynthesizing bacteria, which receive air by microbubbles in quantity according to technical determination, for saturation of the medium and availability to the blend of microalgae that by photosynthesis and processing retain carbon and others gases evidenced by sensory monitoring of CO₂ retention.

While describing the preferred realization of the present one, any modifications and/or alterations must be understood as fitting perfectly within the criteria that define it, that is, the combination and modification of already known elements into a new form or arrangement, resulting in functional improvement in its use or manufacture.

1. A method for air purification by removal of carbon-containing gases from air using photosynthetic microorganisms in an aqueous medium, the method comprising:
(a) preparing a blend comprising microalgae and/or cyanobacteria;
(b) introducing the blend into an aqueous medium contained within at least one photobioreactor;
(c) injecting atmospheric air into the aqueous medium by generating microbubbles using a gas injection subsystem;
(d) dissolving carbon dioxide (CO₂) and carbon monoxide (CO) from the injected air into the aqueous medium;
(e) illuminating the aqueous medium with light having a wavelength in a range of 400 nm to 700 nm to promote photosynthesis;
(f) biologically assimilating the dissolved CO₂ and CO by the microalgae and/or cyanobacteria and releasing oxygen (O₂);
(g) discharging treated air having a reduced concentration of carbon-containing gases; and
(h) monitoring one or more process variables selected from the group consisting of gas concentration, pH, temperature, and biomass density, and adjusting operating conditions to optimize carbon retention efficiency.

2. The method of claim 1, wherein the blend comprises at least one microalga selected from the group consisting of Scenedesmus and Chlorella, and at least one cyanobacterium comprising Spirulina.

3. The method of claim 1, further comprising supplying nutrients to the aqueous medium to sustain growth of the microalgae and/or cyanobacteria.

4. The method of claim 1, wherein the method is performed in a first photobioreactor having a volume of approximately 8 liters for controlled testing of process variables.

5. The method of claim 4, further comprising scaling the method to a second photobioreactor having a volume of approximately 80 liters for validation of organism performance.

6. The method of claim 5, further comprising scaling the method to a third photobioreactor having a volume of approximately 800 liters for large-scale operation and evaluation of system performance.

7. A system for air purification, comprising:
(a) at least one photobioreactor defining a reservoir configured to contain an aqueous medium and a blend of microalgae and/or cyanobacteria;
(b) a gas injection subsystem configured to introduce atmospheric air into the aqueous medium in the form of microbubbles;
(c) an illumination subsystem configured to provide light within a wavelength range of 400 nm to 700 nm;
(d) a monitoring subsystem comprising one or more sensors configured to measure at least gas concentration at an inlet and an outlet of the system, pH, temperature, and humidity; and
(e) an outlet configured to discharge treated air having a reduced concentration of carbon-containing gases.

8. The system of claim 7, wherein the at least one photobioreactor comprises a photobioreactor having a volume of approximately 8 liters.

9. The system of claim 7, wherein the at least one photobioreactor comprises a photobioreactor having a volume of approximately 80 liters.

10. The system of claim 7, wherein the at least one photobioreactor comprises a photobioreactor having a volume of approximately 800 liters.

11. The system of claim 7, wherein the system further comprises a plurality of photobioreactors of different volumes configured for sequential scaling of the air purification process.

12. The system of claim 7, further comprising a display configured to indicate at least one parameter selected from the group consisting of inlet CO₂ concentration, outlet CO₂ concentration, amount of removed CO₂, ambient temperature, and relative humidity.

13. The system of claim 7, further comprising user interface elements including seating structures and electrical outlets.

14. The system of claim 7, wherein the photobioreactor is integrated into a tree-like structure comprising blade elements configured to simulate leaves.

15. The system of claim 14, wherein the blade elements comprise photovoltaic cells.

16. The system of claim 14, wherein the photobioreactor comprises a reservoir formed of a transparent material.

17. The system of claim 7, wherein the photobioreactor has a capacity between 8 liters and 800 liters.

18. The system of claim 7, wherein the photobioreactor has a preferably capacity of 400 liters.
